(19) United States
(12) Patent Application Publication
(10) Pub. No.: US 2025/0209627 A1
(43) Pub. Date: Jun. 26, 2025

(54) DEVICE AND METHOD FOR NON-INVASIVE AND NON-CONTACT PHYSIOLOGICAL WELL BEING MONITORING AND VITAL SIGN ESTIMATION

(71) Applicant: BRGX Technologies LLC, Chattanooga,, TN (US)

(72) Inventors: Pai-chang Yeh, Zhubei (TW); Julian Gerald Dcruz, Kollam (IN); Ted Huang, Taipei (TW)

(73) Assignee: BRGX Technologies LLC, Chattanooga,, TN (US)

(21) Appl. No.: 19/081,519

(22) Filed: Mar. 17, 2025

Related U.S. Application Data
(63) Continuation-in-part of application No. 17/729,523, filed on Apr. 26, 2022.
(63) Continuation-in-part of application No. 17/645,984, filed on Dec. 25, 2021.

(51) Int. Cl. G06T 7/00

(52) U.S. Cl. CPC G06T 7/0014; G06T 2207/10016; G06T 2207/10101; G06T 2207/20081; G06T 2207/20084; G06T 2207/30004; G06T 2207/30201

(57) ABSTRACT

The present disclosure pertains to a non-contact, non-invasive health monitoring device and method utilizing advanced artificial intelligence (AI) and machine learning techniques. This system captures real-time image data of a user's face using a high-resolution camera and processes the data to extract physiological signals, including Photoplethysmography (PPG) and Ballistocardiography (BCG). By leveraging facial landmark detection and deep learning models such as Convolutional Neural Networks (CNNs) and Transformers, the device predicts vital signs such as heart rate, respiratory rate, blood pressure, and oxygen saturation, alongside wellness metrics like stress levels and metabolic health. The device employs robust feature construction and signal processing modules to ensure accurate metrics under varying conditions, with error margins below 5%. Outputs are displayed in real time and integrated with external systems using standardized healthcare protocols. Applications include telehealth, fitness monitoring, public health screening, and automotive safety systems.

### RELATED APPLICATION

This application claims the benefit of U.S. patent application Ser. No. 17/729,523, titled “SYSTEM, METHOD AND APPARATUS FOR NON-INVASIVE & NON-CONTACT MONITORING OF HEALTH CHARACTERISTICS USING ARTIFICIAL INTELLIGENCE (AI)”, filed on Apr. 26, 2022, and to U.S. patent application Ser. No. 17/645/984, titled “APPARATUS, METHOD AND DEVICE FOR NON-CONTACT AND NON-INVASIVE BLOOD SUGAR MONITORING TO HELP MONITOR DIABETIC PATIENTS AND HYPERCOAGULATION”, filed on Dec. 25, 2021. These patent applications are incorporated herein by reference.

### TECHNICAL FIELD

The present disclosure relates to non-contact and non-invasive health monitoring devices. Specifically, the present disclosure involves methods for analyzing physiological signals, including Photoplethysmography (iPPG) and Ballistocardiography (BCG), using deep learning techniques to estimate vital signs and wellness parameters. This present disclosure is particularly applicable to health assessment, fitness monitoring, telehealth, public health screening, and advanced driver assistance systems (ADAS).

### BACKGROUND

The following description includes information that may be useful in understanding the present disclosure. It is not an admission that any of the information provided herein is prior art or relevant to the presently claimed subject matter, or that any publication specifically or implicitly referenced is prior art.

Recent advancements in artificial intelligence (AI) and machine learning have significantly enhanced the capabilities of health monitoring devices, particularly in non-invasive and non-contact methods. Traditional health monitoring devices often require physical contact through wearable sensors or direct physiological measurements, which can cause discomfort and inconvenience for users. These conventional methods may also face limitations in accessibility, particularly in contexts where physical contact is impractical, such as during pandemics, in remote locations, or areas with limited healthcare infrastructure. As a result, there is a growing demand for non-contact solutions that can offer accurate, real-time monitoring without physical interaction.

Non-invasive, video-based monitoring techniques have gained traction for their potential to overcome these limitations. By analyzing subtle changes in physiological signals visible on a person's face, such devices can estimate vital signs and wellness metrics, providing critical health information continuously and unobtrusively. Photoplethysmography (iPPG) and Ballistocardiography (BCG) are two such physiological signals that can be captured and processed to reflect various health metrics. iPPG detects changes in blood volume through light absorption variations in facial regions, while BCG measures micro-movements associated with the heartbeat. Together, these signals provide a rich source of data that, when coupled with advanced machine learning algorithms, can yield valuable insights into a user's health.

Machine learning algorithms, specifically deep learning models, have shown promising results in extracting meaningful information from iPPG and BCG signals. Convolutional Neural Networks (CNNs) are adept at capturing spatial features in these signals, while Transformer-based models with attention mechanisms can analyze temporal patterns. This combination allows for the robust prediction of a range of vital signs, including heart rate, respiratory rate, blood pressure, and blood oxygen saturation. Additionally, wellness parameters such as stress, metabolic health, immune response, and vitamin deficiencies can be estimated using a correlational biocomputational scoring system derived from large datasets.

The ability to offer these health metrics through a non-contact method has significant implications across various industries. In the automotive sector, integrating health monitoring capabilities into Advanced Driver Assistance Systems (ADAS) can enhance driver safety by providing real-time assessments of driver alertness, stress levels, and potential health risks. In public health contexts, non-contact health monitoring devices can facilitate rapid health screenings in high-traffic areas like airports, schools, and workplaces. Furthermore, for fitness enthusiasts and athletes, non-invasive monitoring enables continuous tracking of physiological responses during exercise, supporting optimized training and recovery strategies. Telehealth and remote health monitoring applications also stand to benefit, as these devices enable health assessments from a distance, particularly useful for patients in underserved or rural areas.

Consequently, there is a need for a non-contact, non-invasive solution for real-time health monitoring that performs quickly and accurately.

### SUMMARY

This summary is provided to introduce concepts related to a non-contact, non-invasive health monitoring device and method for monitoring a user's health characteristics using advanced machine learning techniques. The concepts are further described below in the detailed description. This summary is not intended to identify key features or essential features of the claimed subject matter, nor is it intended to be used to limit the scope of the claimed subject matter.

The present disclosure envisages a non-contact, non-invasive health monitoring device. The device includes a camera, a signal processing unit, a prediction unit, an output unit, and a communication interface. The camera is configured to capture real-time image data of a user's face, where the image data including a sequence of frames recorded under a set of conditions that ensure consistent pixel values. The signal processing unit is operatively coupled to the camera. The signal processing unit receives the sequence of frames from the camera. The signal processing unit comprises a video processor, a machine learning accelerator, and a feature construction module. The video processor is implemented as a dedicated hardware circuitry and is configured to detect facial landmarks using a predefined facial landmark detection algorithm and isolate one or more regions of interest (ROIs) based on the detected landmarks to build a time-series sequence of ROI data, where each ROI corresponds to a specific anatomical region. The machine learning accelerator is implemented as a specialized integrated circuit and is configured to receive the time-series sequence of ROI data from the video processor, and apply a trained neural network model, custom trained on annotated video datasets correlated with ground-truth physiological signals selected from the group consisting of, but not limited to: electrocardiogram (ECG), and pulse oximetry data, to extract Photoplethysmography (PPG) and Ballistocardiograph (BCG) signals, representing subtle changes in pixel intensity linked to blood volume pulsations and micro-motions of facial tissue from the temporal and spatial intensity variations within the time-series sequence of ROI data. The feature construction module following the principles of optical computed tomography (OCT) for feature preparation, providing improved background noise and motion reduction allowing better stability of feature extraction in dynamic environments of constant motion, wherein the feature construction module is implemented in hardware logic. The feature construction module is configured to combine the extracted PPG and BCG signals from the machine learning accelerator, with facial image features derived from the time-series sequence of ROI data, wherein said facial image features comprise localized pixel intensity gradients calculated by comparing pixel values within and around the ROI landmarks, thus capturing spatial patterns of intensity variation, and construct a high-dimensional feature representation in the form of a volumetric tensor, where one dimension corresponds to the temporal progression across the frames, effectively treating time as a “depth” dimension, two dimensions represent the spatial coordinates of the ROI, capturing height and width, and one or more additional feature channels represent PPG, BCG, alongside the localized pixel intensity gradients. The prediction unit includes a hardware-based inference engine interfaced to the signal processing unit and is configured to receive the high-dimensional feature representation in the form of a volumetric tensor from the feature construction module; and apply a second trained neural network model, the second trained neural network model is trained on facial video data and corresponding ground-truth physiological measurements, and is made up of a combination of Convolutional layers, and Transformer with self-attention with positional encoding, specialized in analysing the volumetric tensor, combining temporal, spatial, and physiological feature relationships of the PPG, BCG and ROI data of volumetric tensor, to compute at least one physiological metric, with significant improvement in performance with error percentage less than 5% that aligns with medically validated criteria. The output unit comprises a hardware-based display interface configured to display the predicted at least one physiological metric from the prediction unit, in real-time to a user, providing immediate feedback on the subject's health status. The output unit further comprises a hardware-based probabilistic inference component configured to estimate an uncertainty metric associated with the predicted at least one physiological metric by applying a statistically grounded method, and inference using a trained ensemble of models that provides variance estimates, to yield a quantifiable confidence measure indicating the reliability of the prediction. The communication interface is implemented as a hardware module and is configured to transmit the predicted at least one physiological metric and its associated uncertainty metric to an external device or networked system via a standardized communication protocol; and format the transmitted data following standard healthcare data interchange protocols for ensuring compatibility with electronic health record systems or cloud-based analytics platforms.

The present disclosure further envisages a non-contact, non-invasive health monitoring device. The device includes a camera, a signal processing unit, a prediction unit, an output unit, and a communication interface. The camera configured to capture a real-time digital image data of a subject's face. The signal processing unit is operatively coupled to the camera. The signal processing unit receives the real-time digital image data. The signal processing unit comprises video processor, a machine learning accelerator, and a feature construction module. The video processor is implemented as dedicated hardware circuitry and is configured to detect facial landmarks using a predefined facial landmark detection algorithm, and isolate one or more regions of interest (ROIs) based on the detected landmarks to build a real-time series sequence of ROI data, wherein each ROI corresponds to a specific anatomical region. The machine learning accelerator is implemented as a specialized integrated circuit and is configured to receive the real time-series sequence of ROI data from the video processor; and apply a trained neural network mode to the real-time series sequences to extract Photoplethysmography (PPG) and Ballistocardiograph (BCG) signals. The feature construction module following the principles of optical computed tomography (OCT) for feature preparation, providing improved background noise and motion reduction allowing better stability of feature extraction in dynamic environments of constant motion, wherein the feature construction module is implemented in hardware logic. The feature construction module is, configured to combine the extracted PPG and BCG signals from the machine learning accelerator, with facial image features derived from the real-time-series sequence of ROI data, wherein said facial image features comprise localized pixel intensity gradients calculated by comparing pixel values within and around the ROI landmarks, thus capturing spatial patterns of intensity variation; and construct a high-dimensional feature representation in the form of a volumetric tensor, where a first dimension corresponds to the temporal progression across the frames, effectively treating time as a “depth” dimension, a second dimension represents the spatial coordinates of the ROI, capturing height and width, and one or more additional feature channels represent PPG, BCG, alongside the localized pixel intensity gradients. The prediction unit comprises a hardware-based inference engine interfaced to the signal processing unit and is configured to receive the high-dimensional feature representation in the form of a volumetric tensor from the feature construction module; and apply a second trained neural network model, the second trained neural network model is trained on facial video data and corresponding ground-truth physiological measurements, and is made up of a combination of Convolutional layers, and Transformer with self-attention with positional encoding, specialized in analysing the volumetric tensor, combining temporal, spatial, and physiological feature relationships of the PPG, BCG and ROI data of volumetric tensor, to compute at least one physiological metric. The output unit comprises a hardware-based display interface configured to display the predicted at least one physiological metric from the prediction unit, in real-time to a user, providing immediate feedback on the subject's health status. The output unit further comprises a hardware-based probabilistic inference component configured to estimate an uncertainty metric associated with the predicted at least one physiological metric by applying a statistically grounded method, and inference using a trained ensemble of models that provides variance estimates, to yield a quantifiable confidence measure indicating the reliability of the prediction. The communication interface is implemented as a hardware module and is configured to transmit the predicted at least one physiological metric and its associated uncertainty metric to an external device or networked system via a standardized communication protocol; and format the transmitted data following standard healthcare data interchange protocols.

The present disclosure further envisages a method for non-contact, non-invasive health monitoring. The method comprises the steps of capturing real-time digital image data of a subject's face by a camera; receiving the real-time digital image data in a signal processing unit operatively coupled to the camera; detecting facial landmarks using a predefined facial landmark detection algorithm implemented in a video processor; isolating one or more regions of interest (ROIs) based on the detected landmarks to build a real-time series sequence of ROI data, wherein each ROI corresponds to a specific anatomical region; receiving the real-time series sequence of ROI data in a machine learning accelerator; applying a trained neural network model to the real-time series sequence of ROI data to extract Photoplethysmography (PPG) and Ballistocardiograph (BCG) signals; preparing features using a feature construction module based on principles of optical computed tomography (OCT); combining the extracted PPG and BCG signals with facial image features derived from the real-time-series sequence of ROI data, wherein the facial image features comprise localized pixel intensity gradients calculated by comparing pixel values within and around the ROI landmarks to capture spatial patterns of intensity variation; constructing a high-dimensional feature representation in the form of a volumetric tensor, where a first dimension corresponds to the temporal progression across the frames, effectively treating time as a “depth” dimension, a second dimension represents the spatial coordinates of the ROI, capturing height and width, and one or more additional feature channels represent PPG, BCG, and localized pixel intensity gradients; receiving the high-dimensional feature representation in the form of a volumetric tensor in a prediction unit comprising a hardware-based inference engine; applying a second trained neural network model to the high-dimensional feature representation, wherein the second trained neural network model is trained on facial video data and corresponding ground-truth physiological measurements, comprising convolutional layers and Transformer with self-attention and positional encoding, for analyzing the volumetric tensor by combining temporal, spatial, and physiological feature relationships of the PPG, BCG, and ROI data to compute at least one physiological metric; displaying the predicted at least one physiological metric on a hardware-based display interface, in real time, to provide immediate feedback on the subject's health status; estimating an uncertainty metric associated with the predicted physiological metric using a hardware-based probabilistic inference component, by applying a statistically grounded method and inference using a trained ensemble of models that provides variance estimates to yield a quantifiable confidence measure indicating the reliability of the prediction; transmitting the predicted at least one physiological metric and its associated uncertainty metric to an external device or networked system via a communication interface implemented as a hardware module; and formatting the transmitted data according to standard healthcare data interchange protocols.

In an embodiment, the sequence of frames is recorder under controller lighting conditions or dynamically adjusted exposure settings to ensure consistent pixel intensity values.

In an embodiment, the predefined facial landmark detection algorithm is selected from a group consisting of, but not limited to: a Haar cascade classifier, and a deep learning based facial landmark model, stored in on-chip memory, the algorithm identifying reference points such as corners of the eyes, edges of the nostrils, and corners of the mouth.

In an embodiment, the specific anatomical region consisting of, but not limited to: the cheeks, forehead, and the nose, which are known to exhibit minute pixel intensity fluctuations correlated to blood perfusion and micro-movements induced by the cardiovascular and respiratory activity.

In an embodiment, the feature construction module includes a dash camera in vehicles or remote patient bedside monitoring.

In an embodiment, the at least one physiological metric is selected from the group consisting of, but not limited to: pulse rate, breathing rate, and blood oxygen saturation (SpO2), blood pressure, and heart rate variability.

In an embodiment, the statistically grounded method is selected from a group consisting of, but not limited to: Bayesian inference using a prior and likelihood model.

In an embodiment, the standardized communication protocol is selected from a group consisting of, but not limited to: Wi-Fi, Bluetooth, or Ethernet.

In an embodiment, the standard healthcare data interchange protocols are selected from a group consisting of, but not limited to: HL7, FHIR protocols, ensuring compatibility with electronic health record systems or cloud-based analytics platforms.

In an embodiment, the invention can detect spoofing.

The device is equipped to operate across various platforms, including mobile devices, desktops, and cloud-based servers, ensuring wide compatibility. Privacy protection is emphasized through on-device data processing and an optional federated learning approach, where model updates are performed without compromising user privacy. By processing data locally, sensitive health information remains on the user's device, meeting stringent privacy standards such as GDPR (General Data Protection Regulations) and HIPAA (Health Insurance Portability and Accountability Act).

In summary, this device provides a versatile, real-time health monitoring solution that enables non-contact, non-invasive tracking of vital signs and wellness parameters. With applications across automotive safety, public health, fitness, and telehealth, this device represents a significant advancement in health technology, offering reliable, user-friendly, and privacy-conscious health insights for a wide range of users and settings.

Other and further aspects and features of the disclosure will be evident from reading the following detailed description of the embodiments, which are intended to illustrate, not limit, the present disclosure.

### BRIEF DESCRIPTION OF DRAWINGS

The illustrated embodiments of the subject matter will be best understood by reference to the drawings, wherein like parts are designated by like numerals throughout. The following description is intended only by way of example, and simply illustrates certain selected embodiments of devices and methods that are consistent with the subject matter as claimed herein, wherein:

FIG. 1 illustrates an overall architecture of the non-contact health monitoring device, including key components such as the camera, signal processing unit, prediction unit, and output modules, in accordance with an embodiment of the present disclosure;

FIG. 2 illustrates a data flow diagram showing the process of capturing facial video data, extracting physiological signals, and predicting health metrics through machine learning models, in accordance with an embodiment of the present disclosure;

FIG. 3 illustrates mathematical framework and algorithms applied to derive physiological metrics from the high-dimensional feature tensor constructed by the processing unit, in accordance with an embodiment of the present disclosure;

FIG. 4 illustrates the internal operations of the feature construction module, detailing the integration of PPG and BCG signals with pixel intensity gradients, in accordance with an embodiment of the present disclosure;

FIG. 5 illustrates the device integrated with an external monitoring application, highlighting data communication and alert functionalities, in accordance with an embodiment of the present disclosure;

FIG. 6 illustrates the device embedded in automotive setups, such as driver monitoring systems, for real-time health and alert notifications, in accordance with an embodiment of the present disclosure;

FIG. 7 illustrates the integration of the device within fitness equipment for monitoring health metrics during exercise, in accordance with an embodiment of the present disclosure;

FIG. 8 illustrates the device embedded in consumer electronics like televisions, offering wellness monitoring in home environments, in accordance with an embodiment of the present disclosure;

FIG. 9 illustrates the application of the device in telehealth, enabling real-time remote health assessments during virtual consultations, in accordance with an embodiment of the present disclosure; and

FIGS. 10A-10C illustrate a method for non-contact, non-invasive health monitoring in accordance with an embodiment of the present disclosure.

The figures depict embodiments of the disclosure for purposes of illustration only. One skilled in the art will readily recognize from the following description that alternative embodiments of the structures and methods illustrated herein may be employed without departing from the principles of the disclosure described herein.

### DETAILED DESCRIPTION:

A few inventive aspects of the disclosed embodiments are explained in detail below with reference to the various figures. Embodiments are described to illustrate the disclosed subject matter, not to limit its scope, which is defined by the claims. Those of ordinary skill in the art will recognize a number of equivalent variations of the various features provided in the description that follows.

### Non-Limiting Definitions

Definitions of one or more terms that will be used in this disclosure are described below without limitations. For a person skilled in the art, it is understood that the definitions are provided just for the sake of clarity and are intended to include more examples than just provided below.

Camera: A device configured to capture real-time image data of a user's face, comprising a sequence of frames recorded under controlled lighting or dynamically adjusted exposure settings to ensure consistent pixel intensity values.

Signal Processing Unit: A hardware component operatively coupled to the camera, responsible for receiving and analyzing image frames. It includes:

Video Processor: Dedicated circuitry designed to detect facial landmarks and isolate specific Regions of Interest (ROIs).

Machine Learning Accelerator: Specialized circuitry that extracts physiological signals such as Photoplethysmography (PPG) and Ballistocardiography (BCG) using trained neural network models.

Facial Landmark Detection Algorithm: A predefined computational method, such as Haar cascade classifiers or deep learning models, for identifying reference points on the face including eyes, nostrils, and mouth corners.

Regions of Interest (ROIs): Anatomical regions of the face (e.g., cheeks, forehead, nose) exhibiting physiological variations, identified based on detected facial landmarks.

Photoplethysmography (PPG): Represents blood volume changes detected via pixel intensity variations.

Ballistocardiography (BCG): Reflects micro-motions induced by cardiac activity through facial tissue displacements.

Feature Construction Module: Hardware logic implementing optical computed tomography (OCT) principles to combine PPG and BCG signals with facial image features into a high-dimensional feature representation, reducing noise and improving stability.

Volumetric Tensor: A multi-dimensional data structure containing temporal progression of image frames, spatial coordinates of ROIs (height and width), and additional feature channels for PPG, BCG, and pixel intensity gradients.

Prediction Unit: A hardware-based inference engine configured to process volumetric tensors using advanced machine learning models (e.g., CNNs, Transformers) to compute at least one physiological metric.

Output Unit: A display interface providing real-time feedback of physiological metrics.

Probabilistic Inference Component: Provides uncertainty metrics for the predictions using Bayesian inference techniques.

Communication Interface: A hardware module transmitting physiological metrics and uncertainty data to external devices via standardized protocols (e.g., HL7, FHIR, Wi-Fi, Bluetooth).

Physiological Metric: Quantifiable health parameters such as heart rate, respiratory rate, blood pressure, and oxygen saturation, computed with error rates within medically validated criteria.

Standardized Communication Protocols: Protocols ensuring data transmission compatibility, including Wi-Fi, Bluetooth, Ethernet, and healthcare-specific standards like HL7 and FHIR.

Healthcare Data Interchange Standards: Structured frameworks for formatting transmitted health data to ensure integration with electronic health record systems or cloud-based analytics platforms.

### Overview

Traditional health monitoring methods rely on wearable devices or physical contact sensors, which may cause discomfort, restrict movement, and limit usage in certain environments such as public spaces or within vehicles. Additionally, these methods can be impractical or inaccessible in remote areas or situations requiring quick, large-scale health assessments, such as during pandemics or mass gatherings. The challenge lies in achieving reliable, real-time health insights that are convenient, accessible, and suitable for a wide range of users without compromising accuracy.

To solve this problem, the present disclosure introduces an AI-driven device that utilizes high-resolution facial video data to analyze physiological signals and estimate a range of vital signs and wellness parameters. Using a camera to capture facial data, the device processes this data through facial landmark detection and deep learning models to detect Photoplethysmography (iPPG) and Ballistocardiograph (BCG) variations in specific regions of interest. These physiological variations are processed into a high-dimensional feature vector and input into a model combining Convolutional Neural Networks (CNNs) for spatial analysis and Transformer-based attention mechanisms for temporal pattern recognition. This approach allows the device to predict vital signs such as heart rate, blood oxygen levels, and blood pressure, as well as wellness indicators like stress and immune health, without requiring any physical interaction with the user.

The present disclosure signifies advancement in non-contact vital sign estimation permitting the ability to identify liveness and spoof-detection from 1 second with at least 30 frames-per-second (F.P.S) allowing for near real-time analysis for spoof-detection and deepfake analysis. The key identifier being the Photoplethysmography and Balistocardiography variations in specific regions of interest lack periodic nature in AI generated videos and Spoofs resulting in noisy variations, with principal components from the signals lacking correlation to heartbeat like activity. The unique methodology to combine ROIs as a volumetric tensor with the rich information of PPG and BCG allows for near real-time analysis from 1 second videos and provides rich context of information allowing for accurate spoof-detection.

### Exemplary Implementations and Embodiments

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Each of the appended claims defines a separate invention, which for infringement purposes is recognized as including equivalents to the various elements or limitations specified in the claims. Depending on the context, all references below to the “invention” may in some cases refer to certain specific embodiments only. In other cases, it will be recognized that references to the “invention” will refer to subject matter recited in one or more, but not necessarily all, of the claims.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all groups used in the appended claims.

Various embodiments are further described herein with reference to the accompanying figures. It should be noted that the description and figures relate to exemplary embodiments and should not be construed as a limitation to the subject matter of the present disclosure. It is also to be understood that various arrangements may be devised that, although not explicitly described or shown herein, embody the principles of the subject matter of the present disclosure. Moreover, all statements herein reciting principles, aspects, and embodiments of the subject matter of the present disclosure, as well as specific examples, are intended to encompass equivalents thereof. Yet further, for the sake of brevity, operation or working principles pertaining to the technical material that is known in the technical field of the present disclosure have not been described in detail so as not to unnecessarily obscure the present disclosure.

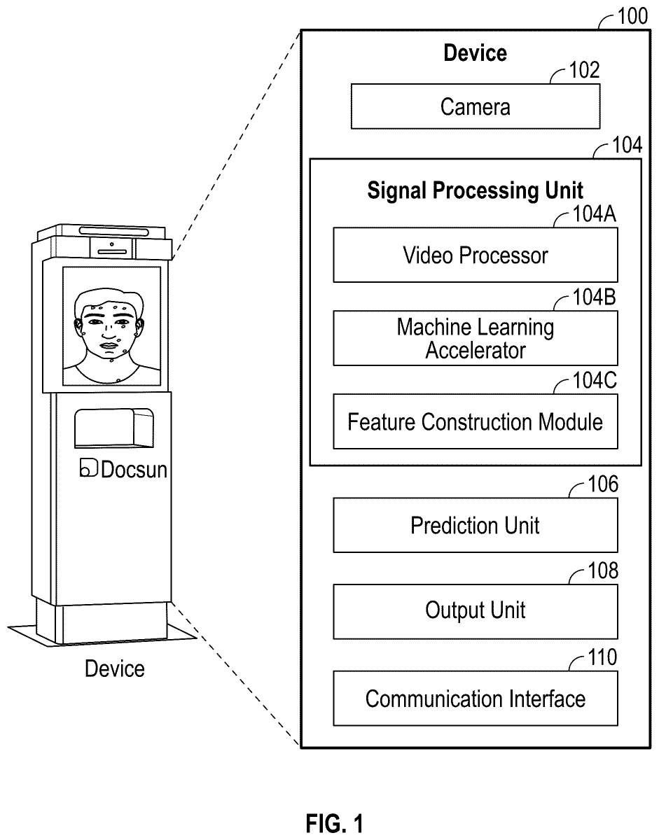

This detailed description provides an in-depth explanation of the device and method employed in the present invention for non-contact, non-invasive health monitoring. The device utilizes a high-resolution imaging unit to capture real-time video data of a user's face, processes this data to detect physiological signals, and employs advanced deep learning techniques to predict a wide range of vital signs and wellness parameters. Key components, their configurations, and interactions within the device will be described, referencing specific elements and functionality based on FIGS. 1 through 13.

As can be seen from FIG. 1, a health monitoring device (100) includes a camera (102), a signal processing unit (104), a prediction unit (106), an output unit (108), and a communication interface (110). Each of these modules interacts to perform the core functions of the invention, which range from image acquisition to real-time health assessment and alert generation.

The device (100) has an architecture that supports integration across various platforms, including mobile, automotive, fitness equipment, and telehealth. The device (100) operates in real time and utilizes federated learning to maintain user privacy while allowing for model updates. The camera (102) is configured to capture real-time image data of a user's face. This image data consists of a sequence of high-resolution frames recorded under controlled lighting conditions or dynamically adjusted exposure settings, ensuring consistent pixel intensity values across frames. These frames are then processed to extract temporal and spatial variations corresponding to physiological signals.

The signal processing unit (104) is operatively coupled to the camera to receive the sequence of frames. The signal processing unit incorporates a video processor (104A), implemented as dedicated hardware circuitry, which detects facial landmarks using predefined algorithms. These algorithms, stored in on-chip memory, may include Haar cascade classifiers or deep-learning-based facial landmark detection models, capable of identifying key reference points such as the corners of the eyes, edges of the nostrils, and corners of the mouth. Using these landmarks, the video processor isolates one or more regions of interest (ROIs) corresponding to specific anatomical regions such as the cheeks, forehead, and nose, which are known to exhibit minute pixel intensity variations associated with blood perfusion and micro-movements due to cardiovascular and respiratory activities.

The time-series data from the ROIs is forwarded to a machine learning accelerator (104B) implemented as a specialized integrated circuit. The machine learning accelerator (104B) is configured to apply a trained neural network model, custom-trained on annotated video datasets correlated with ground-truth physiological signals, such as electrocardiogram (ECG) and pulse oximetry data. This model extracts Photoplethysmography (PPG) and Ballistocardiograph (BCG) signals from the temporal and spatial intensity variations within the ROI data, representing subtle changes in blood volume pulsations and facial micro-motions.

The extracted PPG and BCG signals are further processed in a feature construction module (104C) based on the principles of optical computed tomography (OCT). This module (104C) combines the extracted physiological signals with facial image features derived from localized pixel intensity gradients within and around the ROI landmarks, capturing spatial patterns of intensity variations. The module (104C) constructs a high-dimensional feature representation in the form of a volumetric tensor. This tensor includes temporal progression across frames as one dimension, spatial coordinates of the ROI as two dimensions representing height and width, and additional feature channels representing PPG, BCG, and localized pixel intensity gradients. The feature construction module (104C) is implemented in hardware logic, designed to mitigate background noise and improve motion stability in dynamic environments.

The high-dimensional feature representation is transmitted to a prediction unit (106) comprising a hardware-based inference engine. The prediction unit (106) applies a second trained neural network model, designed using a combination of convolutional layers and Transformer layers with self-attention and positional encoding. This model specializes in analyzing the volumetric tensor by combining temporal, spatial, and physiological feature relationships to compute one or more physiological metrics, such as pulse rate, breathing rate, blood oxygen saturation (SpO2), blood pressure, and heart rate variability. The prediction model is trained to ensure an error margin within medically validated criteria, achieving an error percentage below five percent.

The results of the prediction are presented to the user through the output unit (108). The output unit (108) comprises a hardware-based display interface that provides real-time feedback on the computed physiological metrics, allowing the user immediate insight into their health status. Additionally, a probabilistic inference component in the output unit estimates an uncertainty metric for the prediction. This is achieved by applying a statistically grounded method, such as Bayesian inference using trained ensembles of models, providing a quantifiable confidence measure for the reliability of the prediction.

The device (100) also features the communication interface (110) implemented as a hardware module, enabling the transmission of the physiological metrics and associated uncertainty metrics to external devices or networked systems. The communication interface supports standardized protocols such as Wi-Fi, Bluetooth, or Ethernet, and formats the data to comply with healthcare data interchange standards like HL7 and FHIR protocols, ensuring seamless integration with electronic health record systems or cloud-based analytics platforms. This allows the device (100) to function as part of a broader health monitoring ecosystem, suitable for applications such as remote patient monitoring, vehicle-based health assessments, and real-time health analytics in clinical or home settings.

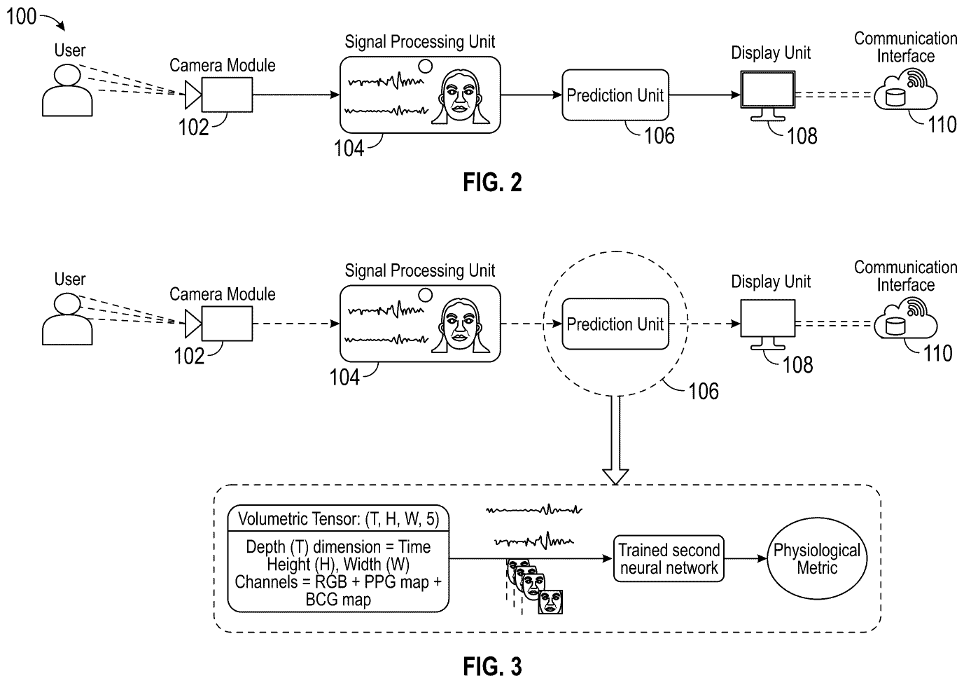

FIG. 2 represents the internal configuration and operation flow of the non-contact, non-invasive health monitoring device (100), detailing the interaction among its primary components. The camera (102) is positioned as the input unit, capturing real-time image data of the user's face. The camera ensures consistency in pixel intensity values by operating under controlled lighting conditions or employing dynamically adjusted exposure settings. The captured image data, comprising a sequence of high-resolution frames, is transmitted to the signal processing unit (104).

Within the signal processing unit, the video processor (104A) detects facial landmarks using predefined algorithms such as Haar cascade classifiers or deep learning models stored in on-chip memory. These landmarks serve as reference points for isolating regions of interest (ROIs) corresponding to anatomical areas like the cheeks, forehead, and nose. The isolated ROIs are analyzed to extract time-series data that captures physiological variations indicative of health metrics.

The time-series ROI data is forwarded to the machine learning accelerator (104B), which applies a trained neural network model. This model, designed specifically for extracting physiological signals, identifies Photoplethysmography (PPG) and Ballistocardiograph (BCG) signals from the temporal and spatial intensity variations within the ROI data. These signals correspond to blood volume pulsations and micro-movements of facial tissues caused by cardiovascular and respiratory activity.

The feature construction module (104C) processes the extracted PPG and BCG signals along with localized pixel intensity gradients derived from the ROI landmarks. By combining these elements, the module creates a high-dimensional feature representation in the form of a volumetric tensor. This tensor encapsulates temporal progression, spatial coordinates, and feature channels representing physiological data, enabling robust analysis under various dynamic conditions.

The constructed tensor is then transmitted to the prediction unit (106), which contains a hardware-based inference engine. Using a trained neural network model composed of convolutional layers and Transformer architecture with self-attention, the prediction unit computes physiological metrics, including pulse rate, breathing rate, blood oxygen saturation (SpO2), and heart rate variability. These metrics are calculated with a medically validated accuracy, ensuring reliable health monitoring.

The output unit (108) displays the computed physiological metrics in real-time, offering immediate feedback to the user. Additionally, the probabilistic inference component estimates an uncertainty metric to provide a confidence measure for the predictions. This ensures that the user or medical practitioner is informed about the reliability of the data.

Finally, the communication interface (110) facilitates the transmission of the computed metrics and associated uncertainty data to external systems or devices. The interface supports standardized communication protocols such as Wi-Fi and Bluetooth and ensures data compatibility with electronic health record systems and cloud-based platforms through compliance with HL7 and FHIR standards. This integrated configuration enables seamless real-time health monitoring and data sharing for enhanced medical care and remote diagnostics.

FIG. 3 illustrates the operational flow and interaction of the signal processing and analysis components within the non-contact, non-invasive health monitoring device (100). The diagram emphasizes the sequential processing of data captured by the camera (102) and its transformation into actionable physiological metrics.

The camera (102) serves as the initial data acquisition module, capturing real-time image data of the user's face under conditions that maintain consistent pixel intensity across frames. The captured image data is sent to the signal processing unit (104), where the video processor (104A) identifies facial landmarks using predefined algorithms. These landmarks delineate specific regions of interest (ROIs) corresponding to anatomical features such as the cheeks, forehead, and nose, which are known to exhibit physiological signals due to blood perfusion and micro-movements caused by cardiovascular and respiratory functions.

The video processor outputs time-series data from the ROIs, which is then processed by the machine learning accelerator (104B). The machine learning accelerator applies a neural network model trained on annotated datasets that correlate video data with ground-truth physiological signals like electrocardiograms (ECG) and pulse oximetry. This processing extracts Photoplethysmography (PPG) and Ballistocardiograph (BCG) signals from temporal and spatial variations in the ROI data. The extracted signals reflect subtle physiological changes such as blood volume pulsations and micro-movements of the facial tissues.

The feature construction module (104C) receives the PPG and BCG signals and combines them with localized pixel intensity gradient features derived from the ROI landmarks. This integration forms a high-dimensional volumetric tensor that preserves temporal, spatial, and intensity-based physiological variations. The tensor's dimensions include temporal progression (time as a depth dimension), spatial coordinates representing the height and width of the ROI, and additional channels for PPG, BCG, and pixel intensity gradients.

The volumetric tensor is passed to the prediction unit (106), which performs advanced analysis using a second neural network model. This model, incorporating convolutional and Transformer-based layers with self-attention, analyzes the tensor to compute physiological metrics such as pulse rate, breathing rate, blood oxygen saturation (SpO2), and heart rate variability. The model ensures high accuracy and aligns with medically validated criteria.

The output unit (108) receives the computed physiological metrics and presents them to the user in real-time through a display interface. In parallel, a probabilistic inference component estimates uncertainty metrics for each prediction, providing a confidence measure for the reliability of the data. This enables informed decision-making by users or medical practitioners.

The communication interface (110) transmits the physiological metrics and their associated uncertainty data to external devices or network systems. This interface supports industry-standard communication protocols like Wi-Fi and Bluetooth and adheres to healthcare data interchange standards such as HL7 and FHIR. FIG. 3 thus showcases the streamlined and robust data flow from initial image capture to real-time physiological analysis and external data integration, highlighting the device's capability to deliver precise and reliable health monitoring.

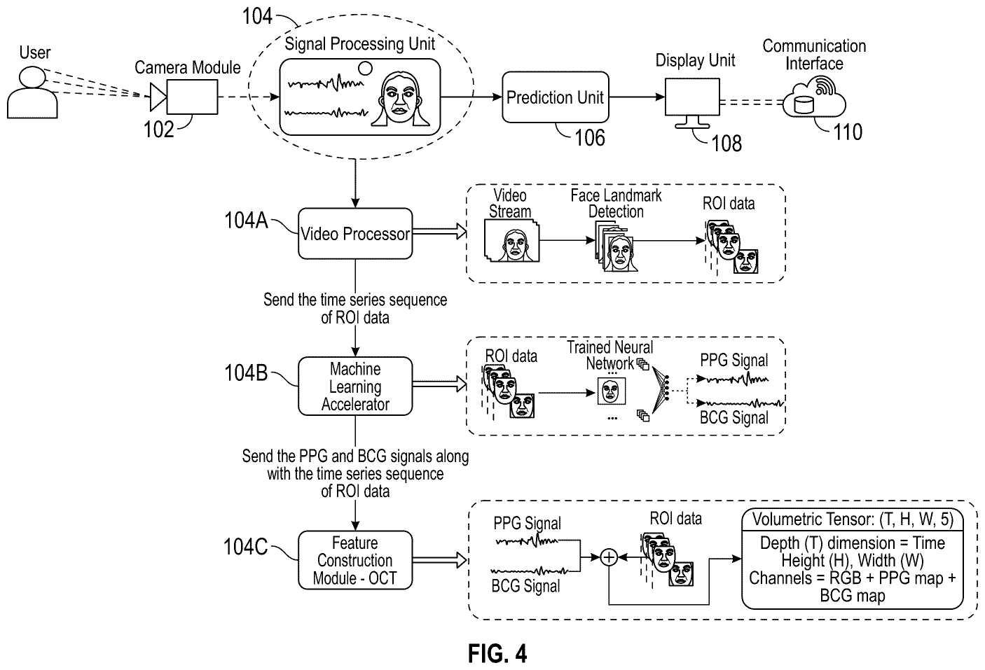

FIG. 4 illustrates the detailed processing workflow within the feature construction module (104C) and its interaction with other core components of the non-contact, non-invasive health monitoring device (100). This figure highlights how the module synthesizes physiological signals and facial image features into a unified, high-dimensional representation for further analysis.

The feature construction module (104C) receives input from the machine learning accelerator (104B) in the form of extracted Photoplethysmography (PPG) and Ballistocardiograph (BCG) signals. These signals, derived from temporal and spatial intensity variations in the ROI data, represent physiological changes associated with blood flow and micro-movements in facial tissue. Simultaneously, the module incorporates localized pixel intensity gradient data computed from the ROI landmarks. These gradients capture spatial patterns of intensity variation around the landmarks, contributing critical information about the facial regions under analysis.

The module utilizes advanced principles of optical computed tomography (OCT) to preprocess the incoming data, reducing background noise and motion artifacts. This ensures that the extracted signals and features remain stable and reliable, even under dynamic environmental conditions such as variable lighting or user motion. The preprocessed data is then combined to construct a volumetric tensor, which serves as a comprehensive representation of the physiological state captured over time.

The volumetric tensor is composed of multiple dimensions. The temporal progression of data frames is represented as the depth dimension, enabling the capture of time-dependent physiological changes. The spatial dimensions correspond to the height and width of the ROIs, preserving the anatomical structure and location-specific features. Additional feature channels within the tensor store the PPG, BCG, and pixel intensity gradient data, ensuring that both physiological and spatial information are fully integrated.

The constructed tensor is passed to the prediction unit (106), as indicated in FIG. 4, for further analysis. The seamless data flow from the feature construction module to the prediction unit ensures that the volumetric tensor retains its high-dimensional structure, enabling the neural network model in the prediction unit to accurately analyze complex temporal and spatial relationships. This modular workflow highlights the robust design of the device, where each processing stage contributes to the accuracy and reliability of the final physiological metrics.

FIG. 4 also underscores the scalability of the feature construction module, which is designed to adapt to various configurations and use cases. For instance, the module can handle data inputs from alternative imaging setups, such as dash cameras in vehicles or remote bedside monitoring devices, while maintaining consistent processing standards. This flexibility makes the module a critical component in extending the applicability of the device across diverse health monitoring environments.

Overall, FIG. 4 provides a comprehensive view of the feature construction module's operations and its central role in bridging raw data acquisition and advanced physiological analysis. By combining cutting-edge data preprocessing techniques with robust feature representation, the module ensures the device's ability to deliver precise and reliable health insights in real-time.

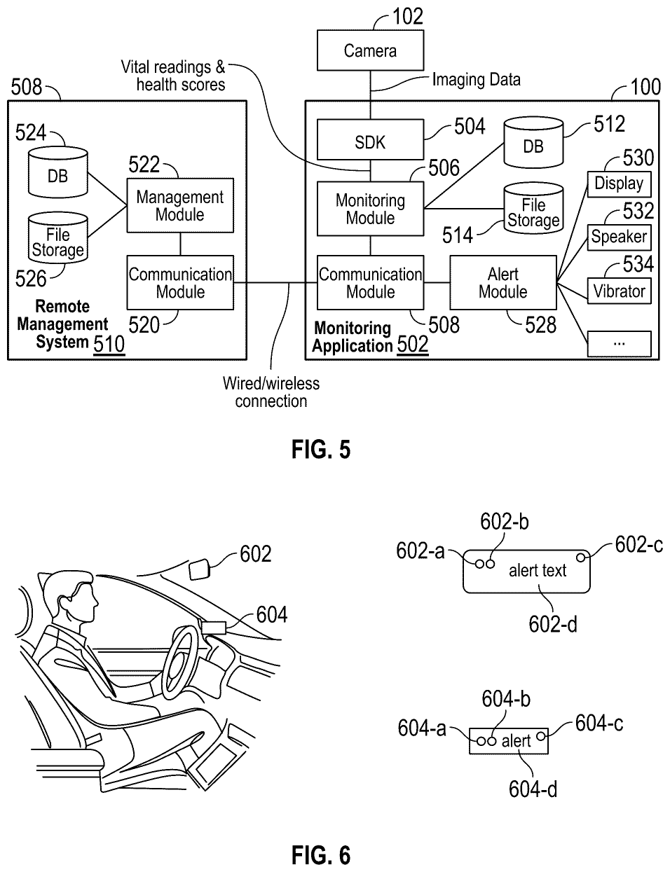

FIG. 5 illustrates an advanced non-contact, non-invasive health monitoring device (100) integrated with a monitoring application (502) and its associated components. At its core, the device (100) includes the camera (102) configured to capture real-time digital image data of a subject's face. This image data is processed through the signal processing unit (104), which houses the video processor (104A) for detecting facial landmarks and isolating regions of interest (ROIs) to generate a time-series sequence of data. The device (100) further includes the machine learning accelerator (104B) that applies a neural network model to extract Photoplethysmography (PPG) and Ballistocardiograph (BCG) signals from the ROI data. These signals represent physiological characteristics such as blood volume pulsations and tissue micro-movements.

The feature construction module (104C) integrates the extracted PPG and BCG signals with localized pixel intensity gradients, constructing a high-dimensional volumetric tensor. This tensor combines temporal data, spatial ROI coordinates, and additional feature channels for advanced analysis. The tensor is passed to the prediction unit (106), which employs convolutional layers and Transformers to compute physiological metrics like pulse rate and oxygen saturation. Results are displayed in real-time via the output unit (108), which also incorporates a probabilistic inference component to estimate the reliability of predictions.

The monitoring application (502) complements this hardware device (100), integrating a Software Development Kit (SDK) (504) for customization, a monitoring module (506) for managing analytics, and a communication module (508) for data exchange with a remote management system (510).

The SDK (504) outputs the vital sign readings to the monitoring module (506). The monitoring module (506) can do active polling to acquire the readings and scores or passively receive the posted data of the readings and scores. The monitoring module (506) will save the readings and scores to a database (DB) (512). The DB (512) and file storage (514) are to keep the records and settings for the monitoring application (502).

The monitoring module (506) contains a logic to check if any reading or score is abnormal or meets the criteria to send an alert. The monitoring module (508) contains configurable alert criteria based on vital sign normal ranges. By default, an alert is triggered by an alert module (528) when any vital sign reading exceeds its normal physiological range. These ranges can be customized by users according to their specific monitoring needs. For example, the heart rate alert threshold could be adjusted for athletes who typically have lower resting heart rates compared to the general population. If sending an alert is required by the alert module (528), the monitoring module (506) will send a signal to the communication module (508) including the alert types to send an alert. The communication module (508) will communicate with the alert module (528) or the communication module (508) through a wired/wireless secure connection, such as HTTPS and/or WebSocket, with the remote management system (510) based on the alert types. The alert module (528) is in charge of sending local alerts such as showing an alert icon or text on the display module (530), playing a notification sound by a speaker (532), triggering a vibrator (534), showing a visual signal via a Light Emitting Diode (LED) light, and so on.

In an embodiment, the communication module (508) of the monitoring application (502) can communicate with a communication module (520) in the remote management system (510) and send over the information of the alert including the reading, score, and identification number of the device (100). In this embodiment, the management module (522) of the remote management system (510) can save the alert information to a database (DB) (524) and file storage (526), then show the alert on a user interface of the remote monitoring system (510). This allows for continuous remote supervision of the user's health, enabling timely intervention when required.

FIG. 6 shows the device (100) built in a car entertainment system, in accordance with an embodiment of the present disclosure. As can be seen from FIG. 6, the camera (102) can be installed at a back mirror (602), a device (604) on top of the dashboard, etc. In one embodiment, the back mirror (602) includes a camera (602-a), an infrared camera (602-b), an audio/light/vibrator alert component (602-c), and a visual text alert (602-d). In another embodiment, the device (604) on the top of the dashboard includes a camera (604-a), an infrared camera (604-b), an audio/light/vibrator alert component (604-c), and a visual text alert (604-d). In both embodiments, the cameras capture the driver's facial features and send them to the real-time SDK to process the signals. The real-time SDK responds to the driver's vital sign values and health scores for the device (100) to track the driver's body status. If the device (100) detects an abnormal situation such as uncomforted, stressed, or abnormal range of the vital signs, the device (100) can alert via visual/audio/vibration to the driver, or send a remote alert to the help center, or trigger the safety protection function of a car such as slowing down the speed, adjusting the temperature of an air conditioner, and so on to mitigate the risks or provide assistance. Alert criteria for driver monitoring can be customized by vehicle manufacturers or fleet operators to set specific thresholds for fatigue detection and stress levels based on their safety protocols.

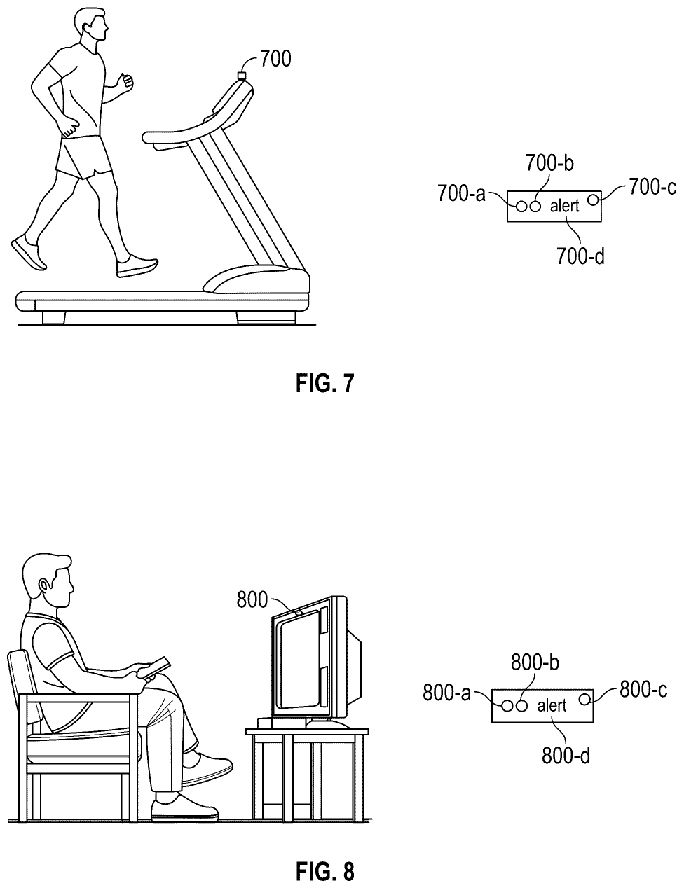

Referring to FIG. 7, the device (100) is integrated within an Advanced Driver Assistance System (ADAS). This setup leverages the real-time SDK and facial imaging data to monitor the physiological state of a driver, providing real-time feedback to enhance driving safety. By detecting signs of fatigue, stress, or other health abnormalities, the device (100) can intervene to mitigate risks associated with impaired driving, thereby reducing the likelihood of accidents caused by driver health issues. The device (100) operates through continuous data capture, analysis, and adaptive feedback mechanisms that interact with the vehicle's safety controls, offering a comprehensive solution for maintaining driver well-being.

FIG. 7 shows the device (100) is implemented as a device (700) built into gym equipment in accordance with an embodiment of the present disclosure. The device (700) is a device attached on top of, or embedded in the gym equipment. The device (700) includes a camera (700-a), an infrared camera (optional) (700-b), an audio/light/vibrator alert component (700-c), and a visual text alert (700-d). The cameras in the device (700) capture the facial features of the user and send them to the real-time SDK or API for analysis. The SDK will respond to vital sign values and health scores to the device (700) for tracking the user's workout and body status. The device (700) can alert via visual/audio/vibration or remote message to a gym help desk when a user is having abnormal issues to avoid overworking and causing injury. In an embodiment, gym equipment manufacturers can customize alert thresholds based on different workout intensity levels, user profiles, specific training programs, or user fitness levels.

Referring to FIG. 7, the device (700) is integrated within fitness monitoring equipment, such as a treadmill, to track the user's physiological metrics continuously during exercise routines. This setup enables the device (700) to provide real-time feedback on the user's physical status, alerting them to any significant changes in health metrics that may indicate overexertion, fatigue, or other health risks. By integrating directly into fitness equipment, the device (700) offers a seamless solution for exercise tracking, allowing users to optimize their workout intensity and ensure safe performance without the need for wearable devices. The device (700) operates by capturing facial imaging data, processing the data through the real-time SDK, and displaying results on the fitness equipment's interface, providing users with instant insights into their physiological state.

FIG. 8 shows the device (100) implemented as a device (800) built into a television set. The device (800) is attached on top of, or a component embedded in the television set. The device (800) includes a camera (800-a), an infrared camera (optional) (800-b), an audio/light/vibrator alert component (800-c), and a visual text alert (800-d). The cameras can be embedded in the television set or a set-top box and capture multiple users' facial features at the same time and send them to real-time SDK or API for analysis. The SDK or API will respond to the vital sign values and health scores of the device (800). The device (800) will keep track of the users' body status and send alerts to the users when there are any abnormal readings or scores to remind the user of further health checks based on the users' profiles. The device (800) can allow healthcare institutions to pre-set custom alert thresholds for different user groups segmented by the user's gender, age, and health conditions. While default alerts are triggered when readings fall outside normal physiological ranges, these thresholds can be adjusted based on individual user conditions and monitoring requirements.

Referring to FIG. 8, the device (800) is integrated into television sets, allowing it to perform non-invasive health monitoring in a home environment. This configuration transforms a common household device into a health and wellness station, enabling users to monitor key physiological metrics while they watch television or relax. The device (800) within the television provides a convenient and unobtrusive way for individuals and families to gain real-time health insights, enhancing awareness of overall wellness and enabling early detection of potential health issues. The television-integrated device (800) captures facial video data, processes it through the real-time SDK, and displays health metrics on-screen, providing users with seamless access to wellness information.

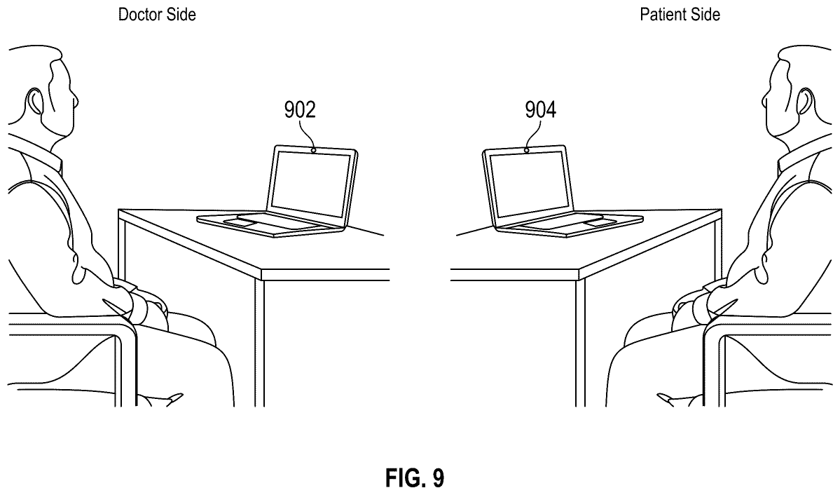

FIG. 9 shows a telehealth device (100) leveraging the computers on the doctor's and patient's sides to capture the patient's facial features in accordance with an embodiment of the present disclosure. In this embodiment, the cameras (902, 904) are implemented in a laptop or computer on the doctor's and patient's sides. The patient can perform a detection before entering a telehealth meeting with the doctor and the doctor will see the patient's vital sign readings and health scores. During the telehealth meeting, the doctor can issue another detection to trigger the detection on the patient's side to acquire the latest readings and scores. The device (100) will alert the doctor if the vital sign readings or health scores are beyond the thresholds set by the healthcare providers. The data will be stored in the telehealth device (100) for the doctor to track the patient's history. Healthcare providers can configure specific alert criteria for different patient conditions. The device (100) comes with default alert thresholds based on standard vital sign ranges, but these can be customized based on patient needs and medical protocols.

Referring to FIG. 9, the device (100) is integrated within telehealth devices to provide non-invasive remote health monitoring capabilities. This setup allows healthcare providers to assess a patient's vital signs and wellness metrics in real-time during virtual consultations. By capturing, processing, and analyzing facial video data, the device (100) offers healthcare professionals a continuous view of the patient's physiological state, enabling prompt and informed decision-making, especially for patients with chronic conditions or limited access to in-person healthcare. The device (100) supports a wide range of health metrics, including heart rate, respiratory rate, blood oxygen saturation (SpO2), and stress levels. By integrating telehealth applications, this setup enhances the quality of remote care and supports proactive health management.

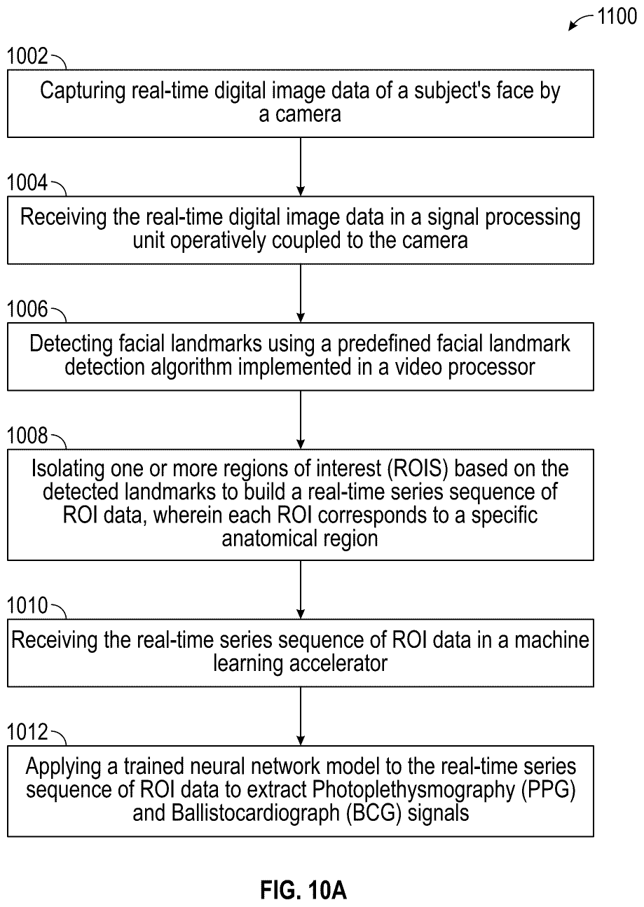

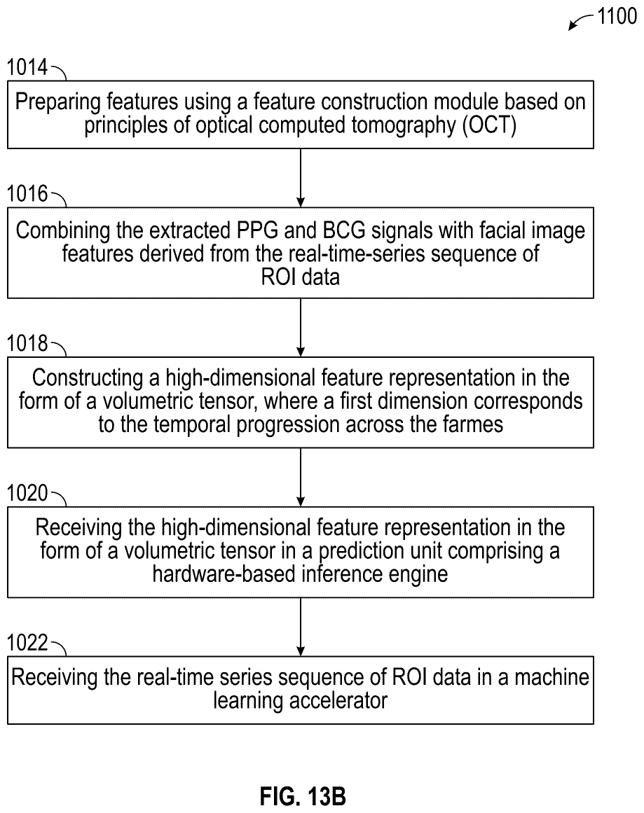

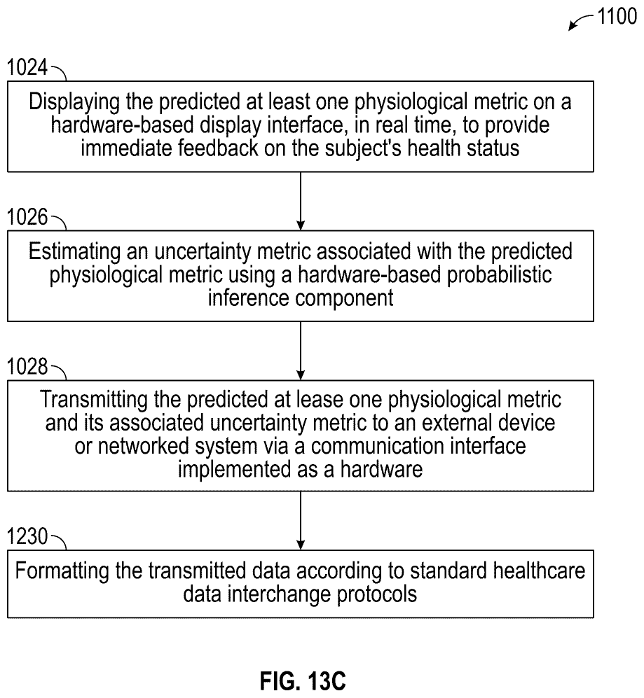

FIGS. 10A-10C illustrate an exemplary method (1000) for non-contact, non-invasive health monitoring, in accordance with an embodiment of the present disclosure. The order in which the method (1000) is described is not intended to be construed as a limitation, and any number of the described method blocks may be combined in any order to implement the methods, or an alternative method. Furthermore, method (1000) may be implemented by processing resource or computing device(s) through any suitable hardware, non-transitory machine-readable instructions, or a combination thereof.

It may also be understood that method (1000) may be performed by programmed computing devices (100) as depicted in FIGS. 1-9. Furthermore, the method (1000) may be executed based on instructions stored in a non-transitory computer-readable medium, as will be readily understood. The non-transitory computer-readable medium may include, for example, digital memories, magnetic storage media, such as one or more magnetic disks and magnetic tapes, hard drives, or optically readable digital data storage media. The method 1000 is described below with reference to computing device(s) as described above; other suitable devices for the execution of these methods may also be utilized. Additionally, the implementation of these methods is not limited to such examples.

At step 1002, the method (1000) begins with capturing real-time digital image data of a subject's face using a camera (102). The camera operates under controlled lighting conditions or dynamically adjusted exposure settings to ensure consistent pixel intensity values.

At step 1004, the real-time image data is received by a signal processing unit (104) that is operatively coupled to the camera (102).

At step 1006, with in the signal processing unit (104), a video processor (104A) implements a predefined facial landmark detection algorithm, identifying facial landmarks such as the corners of the eyes, edges of the nostrils, and corners of the mouth.

At step 1008, the facial landmarks are used to isolate one or more regions of interest (ROIs), corresponding to specific anatomical regions, such as the cheeks, forehead, and nose, which exhibit pixel intensity fluctuations related to physiological signals. This data is organized into a real-time series sequence of ROI data.

At step 1010, a machine learning accelerator (104B) processes this sequence of the ROI data.

At step 1012, the machine learning accelerator (104B) applies a trained neural network model to extract Photoplethysmography (PPG) and Ballistocardiograph (BCG) signals from the ROI data.

At step 1014, a feature construction module (104C), based on principles of optical computed tomography (OCT), prepares features.

At step 1016, the feature construction module (104C), based on principles of optical computed tomography (OCT), prepares features by combining the extracted PPG and BCG signals with localized pixel intensity gradients derived from the ROI data. This integration captures spatial patterns of intensity variation (1114).

At step 1018, the prepared data is structured into a high-dimensional feature representation in the form of a volumetric tensor (1116). This tensor encompasses temporal progression across frames as a “depth” dimension, spatial coordinates representing ROI height and width, and additional feature channels for PPG, BCG, and localized pixel intensity gradients.

At step 1020, the high-dimensional feature representation is received in the form of a volumetric tensor in a prediction unit comprising a hardware-based inference engine.

At step 1022, the volumetric tensor is forwarded to a prediction unit (106), which employs a second neural network model comprising convolutional layers and Transformer architectures with self-attention mechanisms. This model analyzes the tensor to compute at least one physiological metric, such as pulse rate, blood oxygen saturation (SpO2), breathing rate, blood pressure, or heart rate variability (1120).

At step 1024, the prediction is displayed in real-time on a display module (116), providing immediate feedback on the subject's health status. The alert module (118) manages notifications, including visual alerts like icons or text, audible alerts via a speaker (816), tactile feedback using a vibrator (818), and visual signals through an LED light.

At step 1026, a probabilistic inference component estimates an uncertainty metric for each prediction, yielding a confidence measure based on variance estimates derived from a trained ensemble of models (1124).

At step 1028, finally, a communication interface (110) transmits the predicted physiological metrics and uncertainty metrics to an external system, such as a remote management system (510). The data is formatted in compliance with standard healthcare data interchange protocols, ensuring compatibility with electronic health record systems or cloud-based analytics platforms (1128). This method achieves a robust, efficient, and scalable solution for non-contact health monitoring, leveraging advanced hardware and software integration.

The method (1000) offers numerous advantages across clinical, fitness, and autonomous domains through its AI model insights, real-time processing capabilities, and privacy-preserving framework. These advantages ensure that the device proposed herein delivers both accurate health insights and seamless integration into various industries while safeguarding user data.

The AI model utilized in the invention significantly enhances health monitoring in clinical settings by offering a non-contact, non-invasive method to monitor critical vital signs such as include pulse rate, breathing rate, blood oxygen saturation (SpO2), blood glucose level, blood pressure, total cholesterol, heart rate variability, hemoglobin, hematocrit, beta-ketones, and uric acid. By leveraging deep learning techniques and the combination of Photoplethysmography (iPPG) and Ballistocardiograph (BCG) signals, the device provides precise health metrics, reducing the margin of error typically associated with traditional contact-based monitoring devices.

The model's ability to emphasize BCG signals over iPPG signals ensures accurate predictions regardless of skin tone variations and motion artifacts, making it highly reliable for diverse patient populations. Additionally, the Bayesian Linear Layer offers the ability to predict not only vital sign values but also model uncertainty, providing healthcare professionals with confidence levels and reliability indicators for each prediction. This uncertainty estimation can guide clinical decisions, especially in scenarios where accurate real-time data is critical.

Furthermore, the AI model enables continuous monitoring, which is crucial for patients with chronic conditions such as cardiovascular diseases, diabetes, and respiratory disorders. The real-time nature of the model ensures that any sudden changes in a patient's health can be immediately detected, allowing for timely interventions and preventing adverse outcomes.

In the fitness domain, the AI model offers athletes and sports enthusiasts real-time insights into their physiological status, without the need for invasive wearables. The device's ability to predict vital metrics such as heart rate variability, metabolic state, and recovery times allows users to optimize their training routines and avoid overexertion.

Additionally, the AI model helps users track recovery by monitoring wellness parameters such as stress levels, immune health, and bone health. These wellness indicators enable athletes to tailor their fitness programs for maximum performance and recovery, reducing the risk of injury and ensuring balanced training. The continuous, non-invasive nature of the device provides users with uninterrupted feedback on their health, making it a valuable tool for maintaining peak physical performance.

The AI model integrates seamlessly with Advanced Driver Assistance Systems (ADAS) to ensure the safety and well-being of drivers. By analysing real-time video from driver-facing cameras, the device can predict signs of fatigue, stress, or illness that may impair driving ability. The ability to provide real-time feedback to the ADAS system allows for immediate intervention, such as issuing alerts or adjusting vehicle controls, thereby reducing the risk of accidents caused by driver health issues.

The model's fast processing speed and emphasis on BCG signals ensure that accurate health insights are delivered even in dynamic environments like moving vehicles. This capability enhances the safety and performance of autonomous driving systems by continuously monitoring the driver's health and alerting them to any irregularities.

The Software Development Kit (SDK) and Application Programming Interface (API) are designed for real-time processing, offering developers an easy way to integrate the health monitoring device into various platforms, including mobile, automotive, fitness equipment, and telehealth applications. The SDK ensures that the real-time video data captured by the cameras is processed quickly and efficiently, delivering health insights within seconds. This enables immediate action based on the user's health condition, which is particularly useful in time-sensitive situations such as emergency healthcare or driver safety.

The SDK's real-time processing capability is crucial for applications requiring continuous monitoring. For instance, in fitness environments, the device can track a user's physiological responses throughout their workout session, providing instant feedback to help prevent overexertion. Similarly, in telehealth applications, real-time monitoring allows healthcare providers to assess a patient's condition during virtual consultations, ensuring up-to-date health information is available for diagnosis and treatment.

The SDK and API are built with privacy protection in mind. All data processing happens locally on the device, with the option to employ federated learning for AI model updates. This means that user data does not need to be transmitted to centralized servers for processing, significantly reducing the risk of data breaches and ensuring compliance with privacy regulations such as GDPR and HIPAA. By enabling local processing, the device ensures that sensitive health information remains on the user's device, offering users full control over their data.

Additionally, the federated learning approach allows the AI models to be updated over time without compromising user privacy. This ensures that the device continues to improve in accuracy and efficiency while keeping user data secure. The real-time SDK can automatically detect GPU availability or utilize WEBGL acceleration for enhanced processing power, ensuring that users experience fast, secure, and reliable health monitoring regardless of the platform they use.

By combining real-time processing with privacy-preserving techniques, the invention offers a highly secure health monitoring device that caters to various industries while maintaining the highest standards of data protection.

The above description does not provide specific details of the manufacture or design of the various components. Those of skill in the art are familiar with such details, and unless departures from those techniques are set out, techniques, known, related art or later developed designs and materials should be employed. Those in the art are capable of choosing suitable manufacturing and design details.

Note that throughout the disclosure, numerous references may be made regarding servers, services, engines, modules, interfaces, portals, platforms, or other devices formed from computing devices. It should be appreciated that the use of such terms is deemed to represent one or more computing devices having at least one processor configured to or programmed to execute software instructions stored on a computer-readable tangible, non-transitory medium also referred to as a processor-readable medium. For example, a server can include one or more computers operating as a web server, database server, or another type of computer server in a manner to fulfill described roles, responsibilities, or functions. Within the context of this document, the disclosed devices are also deemed to comprise computing devices having a processor and a non-transitory memory storing instructions executable by the processor that cause the device to control, manage, or otherwise manipulate the features of the devices.

It should be understood, however, that all of these and similar terms are to be associated with the appropriate physical quantities and are merely convenient labels applied to these quantities. Unless specifically stated otherwise, as apparent from the discussion herein, it is appreciated that throughout the description, discussions utilizing terms such as “capturing,” or “processing,” or “executing,” or “extracting,” “applying,” “generating,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The exemplary embodiment also relates to an apparatus for performing the operations discussed herein. This apparatus may be specially constructed for the required purposes, or it may comprise a general-purpose computer selectively activated or reconfigured by a computer program stored in the computer. Such a computer program may be stored in a computer-readable storage medium, such as, but is not limited to, any type of disk including floppy disks, optical disks, CD-ROMs, magnetic-optical disks, read-only memories (ROMs), random access memories (RAMs), EPROMS, EEPROMs, magnetic or optical cards, or any type of media suitable for storing electronic instructions, and each coupled to a computer system bus.

Further, the terminology used herein is for the purpose of describing particular embodiments only and is not intended to be limiting to the disclosure. It would be appreciated if several of the above-disclosed and other features and functions, or alternatives thereof, could be combined into other devices or applications. Various presently unforeseen or unanticipated alternatives, modifications, variations, or improvements therein may subsequently be made by those skilled in the art without departing from the scope of the present disclosure as encompassed by the following claims.

The claims, as originally presented and as they may be amended, encompass variations, alternatives, modifications, improvements, equivalents, and substantial equivalents of the embodiments and teachings disclosed herein, including those that are presently unforeseen or unappreciated, and that, for example, may arise from applicants/patentees and others.

It will be appreciated that variants of the above-disclosed and other features and functions, or alternatives thereof, may be combined into many other different devices or applications. Various presently unforeseen or unanticipated alternatives, modifications, variations, or improvements therein may be subsequently made by those skilled in the art which are also intended to be encompassed by the following claims.

What is claimed is:

1. A non-contact, non-invasive health monitoring device, comprising:
a camera configured to capture real-time image data of a user's face, wherein said image data including a sequence of frames recorded under a set of conditions that ensure consistent pixel values;
a signal processing unit operatively coupled to the camera, wherein said signal processing unit receives the sequence of frames from the camera, wherein the signal processing unit comprises:
a video processor implemented as a dedicated hardware circuitry, configured to:
detect facial landmarks using a predefined facial landmark detection algorithm; and
isolate one or more regions of interest (ROIs) based on the detected landmarks to build a time-series sequence of ROI data, wherein each ROI corresponds to a specific anatomical region;
a machine learning accelerator, implemented as a specialized integrated circuit, configured to:
receive the time-series sequence of ROI data from the video processor; and
apply a trained neural network model, custom trained on annotated video datasets correlated with ground-truth physiological signals selected from the group consisting of, but not limited to: electrocardiogram (ECG), and pulse oximetry data, to extract Photoplethysmography (PPG) and Ballistocardiograph (BCG) signals, representing subtle changes in pixel intensity linked to blood volume pulsations and micro-motions of facial tissue from the temporal and spatial intensity variations within the time-series sequence of ROI data;
a feature construction module following the principles of optical computed tomography (OCT) for feature preparation, providing improved background noise and motion reduction allowing better stability of feature extraction in dynamic environments of constant motion, wherein the feature construction module is implemented in hardware logic, configured to:
combine the extracted PPG and BCG signals from the machine learning accelerator, with facial image features derived from the time-series sequence of ROI data, wherein said facial image features comprise localized pixel intensity gradients calculated by comparing pixel values within and around the ROI landmarks, thus capturing spatial patterns of intensity variation; and
construct a high-dimensional feature representation in the form of a volumetric tensor, where:
one dimension corresponds to the temporal progression across the frames, effectively treating time as a “depth” dimension;
two dimensions represent the spatial coordinates of the ROI, capturing height and width; and
one or more additional feature channels represent PPG, BCG, alongside the localized pixel intensity gradients;
a prediction unit, comprising a hardware-based inference engine interfaced to the signal processing unit configured to:
receive the high-dimensional feature representation in the form of a volumetric tensor from the feature construction module; and
apply a second trained neural network model, the second trained neural network model is trained on facial video data and corresponding ground-truth physiological measurements, and is made up of a combination of Convolutional layers, and Transformer with self-attention with positional encoding, specialized in analysing the volumetric tensor, combining temporal, spatial, and physiological feature relationships of the PPG, BCG and ROI data of volumetric tensor, to compute at least one physiological metric, with significant improvement in performance with error percentage less than 5% that aligns with medically validated criteria;
an output unit comprising:
a hardware-based display interface configured to display the predicted at least one physiological metric from the prediction unit, in real-time to a user, providing immediate feedback on the subject's health status; and
a hardware-based probabilistic inference component configured to estimate an uncertainty metric associated with the predicted at least one physiological metric by applying a statistically grounded method, and inference using a trained ensemble of models that provides variance estimates, to yield a quantifiable confidence measure indicating the reliability of the prediction; and
a communication interface, implemented as a hardware module, configured to:
transmit the predicted at least one physiological metric and its associated uncertainty metric to an external device or networked system via a standardized communication protocol; and
format the transmitted data following standard healthcare data interchange protocols for ensuring compatibility with electronic health record systems or cloud-based analytics platforms.

2. The device of claim 1, wherein said sequence of frames is recorded under controller lighting conditions or dynamically adjusted exposure settings to ensure consistent pixel intensity values.

3. The device of claim 1, wherein said predefined facial landmark detection algorithm is selected from a group consisting of, but not limited to: a Haar cascade classifier, and a deep learning based facial landmark model, stored in on-chip memory, the algorithm identifying reference points such as corners of the eyes, edges of the nostrils, and corners of the mouth.

4. The device of claim 1, wherein said specific anatomical region consisting of, but not limited to: the cheeks, forehead, and the nose, which are known to exhibit minute pixel intensity fluctuations correlated to blood perfusion and micro-movements induced by the cardiovascular and respiratory activity.

5. The device of claim 1, wherein said feature construction module includes a dash camera in vehicles or remote patient bedside monitoring.

6. The device of claim 1, wherein said at least one physiological metric is selected from the group consisting of, but not limited to: pulse rate, breathing rate, blood oxygen saturation (SpO2), blood pressure, and heart rate variability.

7. The device of claim 1, wherein said statistically grounded method is selected from a group consisting of, but not limited to: Bayesian inference using a prior and likelihood model.

8. The device of claim 1, wherein said standardized communication protocol is selected from a group consisting of, but not limited to: Wi-Fi, Bluetooth, or Ethernet.

9. The device of claim 1, wherein said standard healthcare data interchange protocols are selected from a group consisting of, but not limited to: HL7, FHIR protocols, ensuring compatibility with electronic health record systems or cloud-based analytics platforms.

10. A non-contact, non-invasive health monitoring device, comprising a camera configured to capture a real-time digital image data of a subject's face;
a signal processing unit, operatively coupled to the camera, where the signal processing unit receives the real-time digital image data, wherein the signal processing unit comprises:
a video processor implemented as dedicated hardware circuitry, configured to:
detect facial landmarks using a predefined facial landmark detection algorithm; and
isolate one or more regions of interest (ROIs) based on the detected landmarks to build a real-time series sequence of ROI data, wherein each ROI corresponds to a specific anatomical region
a machine learning accelerator implemented as a specialized integrated circuit, configured to.
receive the real time-series sequence of ROI data from the video processor; and
apply a trained neural network mode to the real-time series sequences to extract Photoplethysmography (PPG) and Ballistocardiograph (BCG) signals;
a feature construction module following the principles of optical computed tomography (OCT) for feature preparation, providing improved background noise and motion reduction allowing better stability of feature extraction in dynamic environments of constant motion, wherein the feature construction module is implemented in hardware logic, configured to:
combine the extracted PPG and BCG signals from the machine learning accelerator, with facial image features derived from the real-time-series sequence of ROI data, wherein said facial image features comprise localized pixel intensity gradients calculated by comparing pixel values within and around the ROI landmarks, thus capturing spatial patterns of intensity variation; and
construct a high-dimensional feature representation in the form of a volumetric tensor, where:
a first dimension corresponds to the temporal progression across the frames, effectively treating time as a “depth” dimension;
a second dimension represents the spatial coordinates of the ROI, capturing height and width; and
one or more additional feature channels represent PPG, BCG, alongside the localized pixel intensity gradients; and
a prediction unit, comprising a hardware-based inference engine interfaced to the signal processing unit configured to:
receive the high-dimensional feature representation in the form of a volumetric tensor from the feature construction module; and
apply a second trained neural network model, the second trained neural network model is trained on facial video data and corresponding ground-truth physiological measurements, and is made up of a combination of Convolutional layers, and Transformer with self-attention with positional encoding, specialized in analysing the volumetric tensor, combining temporal, spatial, and physiological feature relationships of the PPG, BCG and ROI data of volumetric tensor, to compute at least one physiological metric;
an output unit, comprising:
a hardware-based display interface configured to display the predicted at least one physiological metric from the prediction unit, in real-time to a user, providing immediate feedback on the subject's health status; and
a hardware-based probabilistic inference component configured to estimate an uncertainty metric associated with the predicted at least one physiological metric by applying a statistically grounded method, and inference using a trained ensemble of models that provides variance estimates, to yield a quantifiable confidence measure indicating the reliability of the prediction; and
a communication interface, implemented as a hardware module, configured to:
transmit the predicted at least one physiological metric and its associated uncertainty metric to an external device or networked system via a standardized communication protocol; and
format the transmitted data following standard healthcare data interchange protocols.

11. The device of claim 10, wherein said sequence of frames is recorded under controller lighting conditions or dynamically adjusted exposure settings to ensure consistent pixel intensity values.

12. The device of claim 10, wherein said predefined facial landmark detection algorithm is selected from a group consisting of, but not limited to: a Haar cascade classifier, and a deep learning based facial landmark model, stored in on-chip memory, the algorithm identifying reference points such as corners of the eyes, edges of the nostrils, and corners of the mouth.

13. The device of claim 10, wherein said specific anatomical region consisting of, but not limited to: the cheeks, forehead, and the nose, which are known to exhibit minute pixel intensity fluctuations correlated to blood perfusion and micro-movements induced by the cardiovascular and respiratory activity.

14. The device of claim 10, wherein said feature construction module includes a dash camera in vehicles or remote patient bedside monitoring.

15. The device of claim 10, wherein said at least one physiological metric is selected from the group consisting of, but not limited to: pulse rate, breathing rate, blood oxygen saturation (SpO2), blood pressure, and heart rate variability.

16. The device of claim 10, wherein said statistically grounded method is selected from a group consisting of, but not limited to: Bayesian inference using a prior and likelihood model.

17. The device of claim 10, wherein said standardized communication protocol is selected from a group consisting of, but not limited to: Wi-Fi, Bluetooth, or Ethernet.

18. The device of claim 10, wherein said standard healthcare data interchange protocols are selected from a group consisting of, but not limited to: HL7, FHIR protocols, ensuring compatibility with electronic health record systems or cloud-based analytics platforms.

19. A method for non-contact, non-invasive health monitoring, comprising:
capturing real-time digital image data of a subject's face by a camera;
receiving the real-time digital image data in a signal processing unit operatively coupled to the camera;
detecting facial landmarks using a predefined facial landmark detection algorithm implemented in a video processor;
isolating one or more regions of interest (ROIs) based on the detected landmarks to build a real-time series sequence of ROI data, wherein each ROI corresponds to a specific anatomical region;
receiving the real-time series sequence of ROI data in a machine learning accelerator;
applying a trained neural network model to the real-time series sequence of ROI data to extract Photoplethysmography (PPG) and Ballistocardiograph (BCG) signals;
preparing features using a feature construction module based on principles of optical computed tomography (OCT);
combining the extracted PPG and BCG signals with facial image features derived from the real-time-series sequence of ROI data, wherein the facial image features comprise localized pixel intensity gradients calculated by comparing pixel values within and around the ROI landmarks to capture spatial patterns of intensity variation;
constructing a high-dimensional feature representation in the form of a volumetric tensor, where a first dimension corresponds to the temporal progression across the frames, effectively treating time as a “depth” dimension, a second dimension represents the spatial coordinates of the ROI, capturing height and width, and one or more additional feature channels represent PPG, BCG, and localized pixel intensity gradients;
receiving the high-dimensional feature representation in the form of a volumetric tensor in a prediction unit comprising a hardware-based inference engine;
applying a second trained neural network model to the high-dimensional feature representation, wherein the second trained neural network model is trained on facial video data and corresponding ground-truth physiological measurements, comprising convolutional layers and Transformer with self-attention and positional encoding, for analyzing the volumetric tensor by combining temporal, spatial, and physiological feature relationships of the PPG, BCG, and ROI data to compute at least one physiological metric;
displaying the predicted at least one physiological metric on a hardware-based display interface, in real time, to provide immediate feedback on the subject's health status;
estimating an uncertainty metric associated with the predicted physiological metric using a hardware-based probabilistic inference component, by applying a statistically grounded method and inference using a trained ensemble of models that provides variance estimates to yield a quantifiable confidence measure indicating the reliability of the prediction;
transmitting the predicted at least one physiological metric and its associated uncertainty metric to an external device or networked system via a communication interface implemented as a hardware module; and
formatting the transmitted data according to standard healthcare data interchange protocols.

20. The method of claim 19, wherein said sequence of frames is recorded under controller lighting conditions or dynamically adjusted exposure settings to ensure consistent pixel intensity values.

21. The method of claim 19, wherein said predefined facial landmark detection algorithm is selected from a group consisting of, but not limited to: a Haar cascade classifier, and a deep learning based facial landmark model, stored in on-chip memory, the algorithm identifying reference points such as corners of the eyes, edges of the nostrils, and corners of the mouth.

22. The method of claim 19, wherein said specific anatomical region consisting of, but not limited to: the cheeks, forehead, and the nose, which are known to exhibit minute pixel intensity fluctuations correlated to blood perfusion and micro-movements induced by the cardiovascular and respiratory activity.

23. The method of claim 19, wherein said feature construction module includes a dash camera in vehicles or remote patient bedside monitoring.

24. The method of claim 19, wherein said at least one physiological metric is selected from the group consisting of, but not limited to: pulse rate, breathing rate, blood oxygen saturation (SpO2), blood pressure, and heart rate variability.

25. The method of claim 19, wherein said statistically grounded method is selected from a group consisting of, but not limited to: Bayesian inference using a prior and likelihood model.

26. The method of claim 19, wherein said standardized communication protocol is selected from a group consisting of, but not limited to: Wi-Fi, Bluetooth, or Ethernet.

27. The method of claim 19, wherein said standard healthcare data interchange protocols are selected from a group consisting of, but not limited to: HL7, FHIR protocols, ensuring compatibility with electronic health record systems or cloud-based analytics platforms.

28. The method of claim 19, wherein said standardized communication protocol is used in detecting spoofing.
